(19) United States
(12) Patent Application Publication
(10) Pub. No.: US 2010/0204335 A1
(43) Pub. Date: Aug. 12, 2010

(54) KIT AND COMPOSITION FOR EYELASH GROWTH

(75) Inventors: Frederick C. Beddingfield, Pacific Palisades, CA (US); John G. Walt, Huntington Beach, CA (US); Christine Somogyi, Santa Ana, CA (US); Chetan Pujara, Irvine, CA (US); Julie Albright, Chino, CA (US)

Correspondence Address: ALLERGAN, INC., 2525 DUPONT DRIVE, T2-7H, IRVINE, CA, 92612-1599, US

(73) Assignee: ALLERGAN, INC., Irvine, CA (US)

(21) Appl. No.: 12/627,809

(22) Filed: Nov. 30, 2009

Related U.S. Application Data
(60) Provisional application No. 61/118,841, filed on Dec. 1, 2008.

(51) Int. Cl. A61K 31/5575; A45D 40/26

(52) U.S. Cl. 514/622; 132/218

(57) ABSTRACT

The invention provides a kit for enhancing the appearance of eyelashes, comprising an eyelash enhancing composition and delivery system. The eyelash enhancing composition comprises an effective amount of 0.03% bimatoprost. The delivery system comprises a plurality of applicator brushes designed to deliver a fraction of 1-drop bimatoprost dose to the target area.

### RELATED APPLICATION

This application claims the benefit of U.S. Provisional Application Ser. No. 61/118,841, filed Dec. 1, 2008, the disclosure of which is hereby incorporated in its entirety herein by reference.

### FIELD OF THE INVENTION

Bimatoprost solution 0.03% has been shown to be safe and effective for the following proposed indication:

Bimatoprost solution 0.03% is indicated to improve the prominence of natural eyelashes as measured by increases in growth (length), fullness (thickness), and darkness (intensity).

The proposed dosing regimen is one application nightly directly to the skin of the upper eyelid margin at the base of the eyelashes using the accompanying sterile single-use-per-eye disposable applicators. The proposed packaging and use is included in FIGS. 1 and 2A-B.

### BACKGROUND OF THE INVENTION

Bimatoprost is a synthetic prostaglandin F_{2α} analog, developed by Allergan, Inc. and widely used as an ophthalmic preparation (bimatoprost ophthalmic solution 0.03%) for the treatment of ocular hypertension and open-angle glaucoma. Initial approval of bimatoprost ophthalmic solution 0.03% came from the United States Food and Drug Administration (FDA) in March 2001 and it is currently approved in more than 80 countries worldwide, with approximately 9 million patient-years of exposure worldwide. Initially in clinical trials and later in broad scale use, it became apparent that bimatoprost increases the growth of eyelashes. Based on this observation and after amassing a large safety database on bimatoprost, Allergan developed a clinical program in consultation with the FDA to prospectively assess in a controlled manner the safety and efficacy of bimatoprost solution 0.03% for the improvement of eyelash prominence, length, thickness, and darkness in a healthy adult population.

Bimatoprost for eyelash growth (bimatoprost solution 0.03%) (referred to in this document as BEG) and bimatoprost for the treatment of glaucoma (bimatoprost ophthalmic solution 0.3% marketed as LUMIGAN®) contain the same active product ingredient, in the same formulation (sterile sodium chloride solution in purified water preserved with benzalkonium chloride), at the same concentration (0.03%). Both are applied topically; directly to the eye(s) for the treatment of glaucoma and directly to the upper eyelid margins using a sterile, single-use-per-eye applicator for eyelash growth. Application to the upper eyelid margin via the applicator for eyelash growth delivers approximately 5% of the volume of the drop administered for the treatment of glaucoma.

The safety of bimatoprost is well established, with a large clinical safety database as well as 7 years of postmarketing pharmacovigiliance data. Since the proposed product BEG contains the same active product ingredient, at the same concentration and formulation as bimatoprost for the treatment of glaucoma, and since both are topically applied, the clinical development program for the treatment of glaucoma provides important support for the safety and efficacy of the new drug application (NDA) currently under review.

### Clinical Development Program

Based on the observation of a market need for safe and effective products for enhancing eyelash length, thickness and pigmentation, a clinical development program for an indication of eyelash growth was pursued. The clinical development program was the subject of discussion with the FDA and represents the results of collaboration in the design and execution of studies in this novel area of research.

It was first considered to obtain an indication for eyelash growth with bimatoprost topically applied to the eye. However, to better target drug delivery, direct application of the drug to the base of the upper eyelid margin using a singer-use-per-eye applicator was utilized. An open-label, investigator-sponsored clinical study demonstrated both efficacy and optimized safety with direct application of the reduced amount of bimatoprost to the base of the eyelashes. The efficacy of bimatoprost in growing eyelashes was demonstrated in both phase 3 clinical studies of bimatoprost for the treatment of glaucoma.

### BRIEF DESCRIPTION OF THE DRAWINGS

FIG. 1 shows the proposed packaging of bimatoprost solution 0.03% for eyelash growth; and,

FIG. 2A-B show the brush and application of bimatoprost solution 0.03% for eyelash growth.

### SUMMARY OF THE INVENTION

The safety of bimatoprost 0.03% has been well characterized. Adverse events reported during the pivotal BEG trial were similar to those reported during the clinical development program for glaucoma and were largely localized to the treatment area. The average amount of bimatoprost solution 0.03% delivered to the upper eyelid margins with the applicator was approximately 5% of that delivered by the indicated dose for the treatment of elevated intraocular pressure (IOP) or glaucoma. As would be expected with the considerably lower drug exposure and application solely to the upper eyelid margins used in BEG compared with instillation of eye drops in the LUMIGAN® trials, the observed adverse events with BEG were milder in severity, had a lower rate of occurrence, and rarely resulted in discontinuation from the study.

The adverse events that were most commonly reported by subjects in the bimatoprost-treated group in the pivotal BEG trial were non-serious and predictable based on the known pharmacology of the drug. Consistent with the lower level of exposure compared with treatment of elevated IOP, they were also more likely to be mild in severity. The following adverse events were observed: eye pruritus (5/137, 3.6%), conjunctival hyperemia (5/137 3.6%), skin hyperpigmentation (4/137, 2.9%), pinguecula (3/137, 2.2%), eye irritation (3/137, 2.2%), dry eye (3/137, 2.2%), and erythema of eyelid (3/137, 2.2%). Only 4 of 137 subjects in the bimatoprost group discontinued from the study due an adverse event. Conjunctival hyperemia was the only adverse event reported at a statistically significantly higher rate than vehicle (3.6% versus 0.0%, p=0.028); however, the incidence of conjunctival hyperemia after 4-months of treatment in the pivotal BEG trial was much lower than that observed in trials of LUMIGAN® instilled as a topical ophthalmic for the treatment of elevated IOP (38.4% to 51.8% for QD and BID administration, respectively, after 4 months of treatment in IOP studies).

### Efficacy Results

Bimatoprost solution 0.03% was found to be highly effective. All of the objectives of the pivotal trial were successfully achieved—bimatoprost-treated subjects experienced greater improvements than vehicle-treated subjects in the measurements of eyelash prominence, length, thickness, and darkness, to a highly statistically significant degree (p<0.001 for each endpoint).

A statistically significantly higher percentage of subjects in the bimatoprost group compared with the vehicle group experienced improved eyelash prominence (defined as at least a 1-grade increase on the 4-point Global Eyelash Assessment [GEA] scale), length, thickness, and darkness (p<0.0001 for each endpoint). While the primary time point for efficacy was identified a priori as the week 16 visit, statistically significant differences between the bimatoprost group and vehicle group were first observed at week 8 for the primary endpoint of eyelash prominence, at week 4 for the secondary endpoint of eyelash length, and at week 8 for the secondary endpoints of eyelash thickness and darkness. For all endpoints, the difference between the 2 groups became progressively more pronounced with continued treatment and was highly statistically significant at all subsequent time points during the treatment period. The effects of improved eyelash prominence, length, thickness, and darkness continued to be evident to a statistically significant degree in the bimatoprost group as compared to vehicle through the 1-month posttreatment follow-up period.

In the evaluation of PROs, subjects in the bimatoprost group as compared with the vehicle group reported a statistically significantly higher degree of satisfaction with their eyelashes in terms of the physical attributes of their eyelashes (i.e., satisfaction with eyelashes in terms of length, fullness, and overall satisfaction), the subjective attributes of their eyelashes (i.e., satisfaction with eyelashes as they relate to feelings of confidence, professionalism, and attractiveness), and the daily routine of making their eyelashes presentable. These results indicate that the eyelash growth experienced by subjects in the bimatoprost group was aesthetically meaningful in terms of their overall satisfaction with their eyelashes.

Bimatoprost solution 0.03%, applied topically to the upper eyelid margins at a dose that is approximately 5% of the recommended ophthalmic dose of LUMIGAN®, is safe and effective in improving the prominence of natural eyelashes as measured by increases in growth (length), fullness (thickness), and darkness (intensity). If approved, this product will be the first eyelash enhancement product to be developed under FDA guidance and manufactured under good manufacturing practices, safely satisfying a desired aesthetic need in the marketplace. Given the substantial clinical and postmarketing safety data with bimatoprost ophthalmic solution and the positive results from the pivotal trial for bimatoprost for eyelash growth, it is believed that bimatoprost solution 0.03% can provide patients with meaningful aesthetic benefits with minimal risk. As a prescription drug product with an approved label and risk minimization plan, prescribers and patients would be assured of the safety and efficacy of BEG, which cannot be similarly confirmed for some over the counter or unapproved eyelash treatments.

Some embodiments of the invention include:
1) A kit for enhancing the appearance of eyelashes, comprising: an eyelash enhancing composition and a delivery system, wherein the eyelash enhancing composition comprises an effective amount of 0.03% or 0.01% bimatoprost solution and wherein the delivery system comprises a plurality of applicator brushes having filaments designed to deliver a fraction of a 1-drop bimatoprost dose directly to the target treatment area.
2) The kit of paragraph 1, wherein the delivery system controls the rate of release of the composition from the brush to an eye surface.
3) The kit of paragraphs 1 and 2 wherein the delivery system delivers a bimatoprost dose which is 5% of the bimatoprost dose delivered by 1 drop of bimatoprost for treatment of glaucoma or lowering IOP.
4) The kit of any of the paragraphs 1-3, wherein the target treatment area is the upper eyelid margin.
5) The kit of any of the paragraphs 1-4, wherein the applicator brush is sterile.
6) The kit of any of the paragraphs 1-5, wherein the applicator brush is disposable.
7) The kit of any of the paragraphs 1-6, wherein the composition is disposed within a dispensing container.
8) The kit of any of the paragraphs 1-7, wherein the dispensing container is squeezable.
9) The kit of any of the paragraphs 1-8, wherein a maximum of 2 or 3 or 4 or 5 or 6 ml of the composition is disposed in the dispensing container.
10) The kit of any of the paragraphs 1-9, wherein the applicator brushes are packaged in a plastic sheath.
11) The kit of any of the paragraphs 1-10, wherein the applicator brushes are packaged in sets of two or sets of four.
12) The kit of the paragraphs 1-11, wherein the kit contains 60 or 120 applicator brushes.
13) The kit of any of the paragraphs 1-12, further comprising instructions for using the eyelash enhancing composition and delivery system.
14) The kit of any of the paragraphs 1-13, wherein the delivery system, composition and instructions are enclosed in a box.
15) The kit of any of the paragraphs 1-14, wherein the container of the composition and the instructions are contained in a first box, and wherein the delivery system is contained in a second box and wherein the first box and second box are contained together in a third box.
16) A method of enhancing the growth of eyelashes wherein the amount of bimatoprost applied a per eyelid margin is 5-12 μg/drop of bimatoprost.
17) The method of paragraph 16 wherein the amount of bimatoprost applied per eyelid margin per day is 9 μg/drop of bimatoprost.
18) The method of any of the paragraphs 1-17 wherein the amount of 0.03% bimatoprost applied per eyelid margin is 2-8% of a 30 μL dose of 0.03% bimatoprost.
19) The method of any of the paragraphs 1-18 wherein the amount of 0.03% bimatoprost applied per eyelid margin is 3-7% of a 30 μL dose of 0.03% bimatoprost.
20) The method of any of the paragraphs 1-19 wherein the amount of 0.03% bimatoprost applied per eyelid margin is 4-6% of a 30 μL dose of 0.03% bimatoprost.
21) The method of any of the paragraphs 1-20 wherein the amount of 0.03% bimatoprost applied per eyelid margin is 5% of a 30 μL dose of 0.03% bimatoprost.

### DETAILED DESCRIPTION OF THE INVENTION

The total dose delivered and therefore the total systemic exposure to bimatoprost with topical application to the upper eyelid margins for the enhancement of eyelash growth are much lower than those for LUMIGAN® ophthalmic solution for the treatment of elevated IOP or glaucoma. In the use of bimatoprost for the treatment of glaucoma, a drop of bimatoprost ophthalmic solution is instilled directly into the eye leading not only to eye exposure but also eyelid skin and eyelash exposure via a bathing of the eyelid margin and eyelashes in the bimatoprost solution.

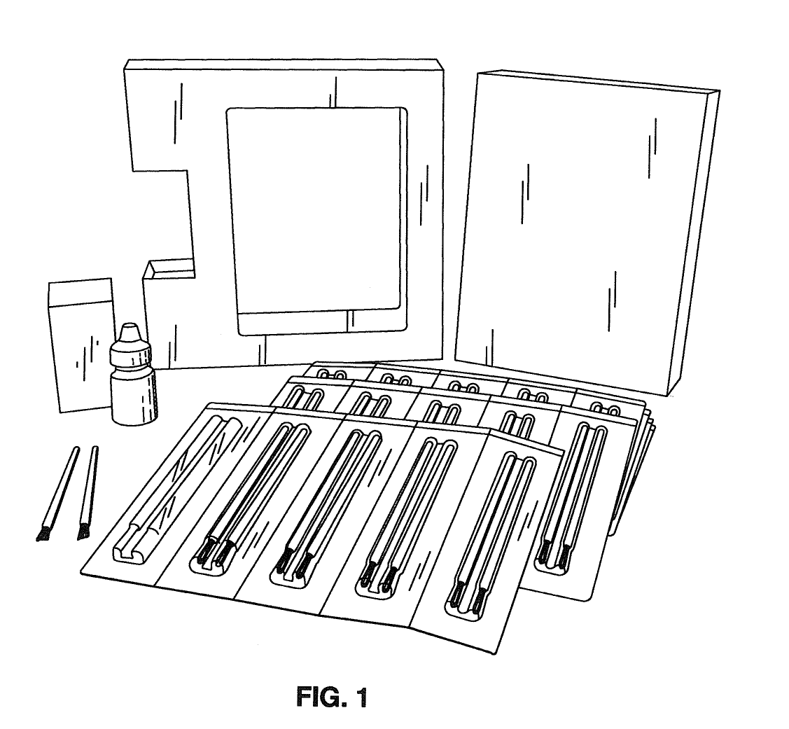

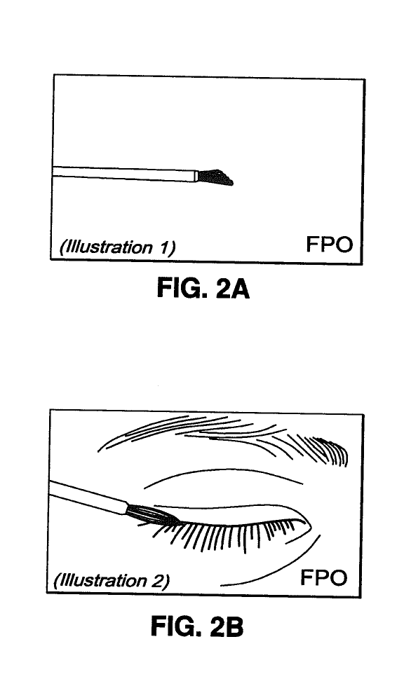

The BEG applicator (see FIGS. 1 and 2) was designed to deliver a fraction of a 1-drop bimatoprost dose directly to the target treatment area. With a single BEG application, approximately 5% of the dose for the treatment of glaucoma is delivered to the upper eyelid margin. The subsequent absorption of bimatoprost from the eyelid surface into the ocular tissues and the body is expected to be incomplete due to the protective skin barrier due to the small surface area upon which the dose is applied.

The recommended daily dose of Lumigan is one 30 μL eyedrop applied topically to each eye once daily. The bimatoprost dose contained in each eyedrop is 9 μg, calculated as follows.

0.03%=0.03 g/100 mL=30 mg/100 mL=30 μg/100 μL

X=30 μL drop*30 μg/100 μL=9 μg/drop

With a single BEG application, approximately 5% of this dose is delivered on average to the upper eyelid margin. The subsequent absorption of bimatoprost from the eyelid surface into the ocular tissues and the body is expected to be incomplete due to the protective skin barrier and due to the small surface area upon which the dose is applied (Dugard, 1986; Trommer and Neubert, 2006; Steiling et al, 2001).

Other dosing ranges include 0.01-1, 1-9, 2-8%, 3-7%, 4-6% or 5% of a dose of 30 μL LUMIGAN or 0.03% bimatoprost or 1-12, 2-11, 3-10, 4-9, 5-9, 5-9, 6-11, 7-10, 8-10 or 9 μg/drop of bimatoprost or approximately 1.5 μg/drop.

Systemic exposure was measured after a 1-drop administration of bimatoprost ophthalmic solution 0.03% to both eyes of 15 healthy subjects once daily for 2 weeks using a state-of-the-art sensitive Liquid Chromatography Coupled with Mass Spectroscopy (LCMS) method during the development of bimatoprost ophthalmic solution 0.03%. The mean Cₘₐₓ values were similar on days 7 and 14 at approximately 0.08 ng/mL, which was approximately 3 times the lower limit of quantitation of the LCMS method. Because the BEG applicator only transfers a small fraction of the bimatoprost dose onto the eyelid margins (approximately 5%), the systemic exposure of bimatoprost from the BEG application would not have been measurable using this sensitive method.

Instructions for use once a day in the evening:
1. Start by ensuring your face is clean, makeup and contact lenses are removed, and any other facial care products have been applied.
2. Remove an applicator from its tray. Then, holding the sterile applicator horizontally, apply one drop of TRADENAME™ to the area of the applicator closest to the tip but not on the tip.
3. Then immediately draw the applicator carefully across the skin of the upper eyelid at the base of the eyelashes (where the eyelashes meet the skin) going from the inner part of your eyelash line to the outer part (see FIG. 2). This area should evenly and lightly moist without runoff.
4. Blot any excess solution beyond the eyelid margin.
5. Dispose of the applicator after one use. Repeat the opposite eyelid margin using a new sterile applicator. This helps minimize any potential for contamination from one eyelid to another.

Systemic, ocular, and eyelid exposures to bimatoprost after ophthalmic administration of 1 drop of LUMIGAN® have been demonstrated to be safe through extensive nonclinical and clinical studies and by 7 years of postmarketing surveillance. Specific to eyelid tissues, nonclinical pharmacokinetic research shows that a substantial portion of the ophthalmic bimatoprost dose is absorbed by the eyelid tissues. The total eyelid exposure following BEG application is expected to be less than that following ophthalmic dosing of bimatoprost to the eyes, which is well tolerated. Because of this, the safety of the BEG dose is well supported by extensive safety data from nonclinical, clinical, and postmarketing experience with LUMIGAN®.

The safety of bimatoprost for the enhancement of eyelash growth has been demonstrated by the favorable adverse event profile observed in the pivotal BEG trial. In the pivotal BEG trial, bimatoprost solution 0.03% was applied topically to the upper eyelid margins of healthy adult subjects, at a dose approximately 5% of that of a 1-drop dose of LUMIGAN® for the treatment of elevated IOP. As would be expected with the considerably lower exposure from BEG administration as compared with LUMIGAN® administration, adverse events reported during the pivotal BEG trial occurred at a low frequency and were largely mild in severity, aesthetic in nature, and reversible. Adverse events did not usually lead to discontinuation from the study. Importantly, patient satisfaction with bimatoprost treatment was generally not impacted by the experience of an adverse event. As shown in Table I, 72.5% of bimatoprost-treated subjects who experienced any adverse event during the pivotal BEG trial still reported satisfaction with their eyelashes at the end of the treatment period. Interestingly, subjects who did not experience an adverse event reported satisfaction with their eyelashes at a slightly lower rate (60.5%). In terms of the mean change from baseline in scoring of overall satisfaction with eyelashes, bimatoprost-treated subjects who experienced an adverse event reported improved satisfaction of more than 2 points on a 5-point scale (possible answers were very dissatisfied, dissatisfied, neutral, satisfied, and very satisfied), indicating that the experience of an adverse event did not impact the subjects' perception of a benefit from bimatoprost.

TABLE I
Impact of the Experience of an Adverse
Event on Patient Reported Outcomes

| | Bimatoprost Solution 0.03% for Eyelash Growth | |
|---|---|---|
| | Subjects Reporting | Subjects Not Reporting |
| | Adverse Event(s) | Adverse Event(s) |
| Subjects feeling | 37/51 (72.5) | 52 (60.5) |
| satisfied or very | | |
| satisfied at Week | | |
| 16^{a}, N (%) | | |
| Change from baseline | −2.04 (−1.33) | −1.80 (−1.21) |
| to week 16 | | |
| on overall satis- | | |
| faction^{b}, Mean (SD) | | |

SD = standard deviation
^{a}Week 16 marked the end of the treatment period in the pivotal BEG study (192024-032).
^{b}The question, “Overall, how satisfied are you with your eyelashes?” was item #4 on the PRO questionnaire. Subjects answered the question using a 5-point scale; possible answers were very dissatisfied, dissatisfied, neutral, satisfied, or very satisfied. A negative change from baseline indicated an increase in satisfaction.

Certain “class effects” commonly associated with prostaglandin F₂ analogs, such as skin hyperpigmentation, hair growth outside the treatment area, hyperemia, and iris hyperpigmentation, were reported in the BEG trial by very few subjects. With few exceptions (2 subjects reporting skin hyperpigmentation and 1 subject reporting errant hair growth outside the treatment area), all of these events had resolved prior to the end of the study. IOP reduction, while statistically significantly different between the bimatoprost and vehicle groups, was minimal (i.e., less than 1 mm Hg difference in mean IOP changes from baseline between the 2 treatment groups at any time point during the study) and thus, was not clinically significant.

BEG is an aesthetic product. Therefore, its benefits must be considered first from the point of view of the patient. The benefits of bimatoprost solution 0.03% have been clearly demonstrated in the pivotal BEG trial, not only through the clinical measurements of prominence, length, thickness, and darkness, but by the greater increases in satisfaction reported by subjects in the bimatoprost group versus the vehicle group. PRO data indicate that, compared with vehicle-treated subjects, subjects in the bimatoprost group were significantly more satisfied with the physical (e.g., length, fullness) and subjective (e.g., confidence, attractiveness) attributes of their eyelashes, as well as with their eyelashes overall. These results clearly show that the benefits of bimatoprost for eyelash growth are not only noticeable through the statistical interpretation of clinical measurements but are noticeable and appreciated by the persons who use the product. In line with these PRO results, quantitative improvements in eyelashes were demonstrated in the pivotal BEG trial by the significant efficacy of bimatoprost compared with vehicle in the clinical measurements of prominence, length, thickness, and darkness. By the end of the 16-week treatment period, 78.1% of subjects in the bimatoprost group had experienced improved eyelash prominence compared with only 18.4% of subjects in the vehicle group. Subjects in the bimatoprost group had experienced percentage increases in eyelash length, thickness, and darkness of 25%, 106%, and 18%, respectively, while subjects in the vehicle group experienced only 2%, 12%, and 3% increases in eyelash length, thickness, and darkness, respectively.

### CONCLUSION

Bimatoprost solution 0.03%, applied topically to the upper eyelid margins, at approximately 5% the dose used for lowering IOP or treatment of glaucoma, has been conclusively shown to be safe and effective in improving the prominence of natural eyelashes as measured by increases in growth (length), fullness (thickness), and darkness (intensity), thus providing a key benefit desired by consumers. The safety database for bimatoprost is substantial, with clinical trial exposure to bimatoprost estimated at 3461 patient-years and worldwide postmarketing exposure estimated at 9 million patient-years. Bimatoprost ophthalmic solution 0.03% has been used safely and successfully for over 7 years in a large multi-ethnic population around the world. The pivotal trial for bimatoprost for eyelash growth confirmed the highly favorable safety profile that was expected for bimatoprost 0.03% when applied topically to the upper eyelid margins at a dose approximately 5% of the indicated 1-drop LUMIGAN® dose for the treatment of glaucoma. In addition to the favorable safety profile demonstrated in the pivotal BEG trial, excellent efficacy was observed for all endpoints, with differences between bimatoprost and vehicle reaching high statistical significance for the measurements of eyelash prominence, length, thickness, and darkness (p<0.0001 for each endpoint at the primary time point). Patient-reported outcomes results clearly showed that the benefits of bimatoprost for eyelash growth are not only noticeable through the statistical interpretation of clinical measurements, but are noticeable and appreciated by the persons who use the product.

Given the long history of clinical and postmarketing safety with bimatoprost ophthalmic solution and the positive results from the pivotal trial for bimatoprost for eyelash growth, it is clear that bimatoprost solution 0.03% can provide meaningful aesthetic benefit to the patients who use it while posing minimal risk. If approved, this product will be the first eyelash enhancement product to be developed under FDA guidance and manufactured under good manufacturing practices. Furthermore, the launch of this product with comprehensive labeling for use under physician supervision and a risk minimization plan including enhanced pharmacovigilance, will further ensure the safe use of the product in the marketplace and allow patient access to a highly desired aesthetic benefit.

What is claimed:

1) A kit for enhancing the appearance of eyelashes, comprising: an eyelash enhancing composition and a delivery system, wherein the eyelash enhancing composition comprises an effective amount of 0.03% bimatoprost solution and wherein the delivery system comprises a plurality of applicator brushes having filaments designed to deliver a fraction of a 1-drop bimatoprost dose directly to the target treatment area.

2) The kit of claim 1, wherein the delivery system controls the rate of release of the composition from the brush to an eye surface.

3) The kit of claim 1 wherein the delivery system delivers a bimatoprost dose which is 5% of the bimatoprost dose delivered by 1 drop of bimatoprost for treatment of glaucoma or lowering IOP.

4) The kit of claim 1, wherein the target treatment area is the upper eyelid margin.

5) The kit of claim 1, wherein the applicator brush is sterile.

6) The kit of claim 1, wherein the applicator brush is disposable.

7) The kit of claim 1, wherein the composition is disposed within a dispensing container.

8) The kit of claim 7, wherein the dispensing container is squeezable.

9) The kit of claim 7, wherein a maximum of 3 ml of the composition is disposed in the dispensing container.

10) The kit of claim 1, wherein the applicator brushes are packaged in a plastic sheath.

11) The kit of claim 1, wherein the applicator brushes are packaged in sets of two.

12) The kit of claim 1, wherein the kit contains 60 applicator brushes.

13) The kit of claim 1, further comprising instructions for using the eyelash enhancing composition and delivery system.

14) The kit of claim 13, wherein the delivery system, composition and instructions are enclosed in a box.

15) The kit of claim 14, wherein the container of the composition and the instructions are contained in a first box, and wherein the delivery system is contained in a second box and wherein the first box and second box are contained together in a third box.

16) A method of enhancing the growth of eyelashes wherein the amount of bimatoprost applied a per eyelid margin is 5-12 μg/drop of bimatoprost.

17) The method of claim 16 wherein the amount of bimatoprost applied per eyelid margin per day is 9 μg/drop of bimatoprost.

18) The method of claim 17 wherein the amount of 0.03% bimatoprost applied per eyelid margin is 2-8% of a 30 μL dose of 0.03% bimatoprost.

19) The method of claim 17 wherein the amount of 0.03% bimatoprost applied per eyelid margin is 3-7% of a 30 μL dose of 0.03% bimatoprost.

20) The method of claim 17 wherein the amount of 0.03% bimatoprost applied per eyelid margin is 4-6% of a 30 μL dose of 0.03% bimatoprost.

21) The method of claim 17 wherein the amount of 0.03% bimatoprost applied per eyelid margin is 5% of a 30 μL dose of 0.03% bimatoprost.
